(19) United States
(12) Patent Application Publication
(10) Pub. No.: US 2009/0280199 A1
(43) Pub. Date: Nov. 12, 2009

(54) Neuroprotective Compositions and Methods of Using Same

(75) Inventor: Michael J. Russell, Davis, CA (US)

Correspondence Address: MORGAN, LEWIS & BOCKIUS LLP (SF), One Market, Spear Street Tower, Suite 2800, San Francisco, CA, 94105, US

(73) Assignee: AAKEN LABORATORIES, INC., DAVIS, CA (US)

(21) Appl. No.: 12/264,252

(22) Filed: Nov. 3, 2008

Related U.S. Application Data
(60) Provisional application No. 60/984,665, filed on Nov. 1, 2007.

(51) Int. Cl. A61K 36/45; A61P 25/00

(52) U.S. Cl. 424/732

(57) ABSTRACT

The present invention provides compositions and methods for preconditioning, providing neuroprotection to, and/or enhancing the neural recovery of an individual. The compositions include pomegranate extract, blueberry extract, curcumin and/or magnesium citrate, and Omega 3 fatty acids. The compositions can be used in beverages or food products. In preferred embodiments, the compositions are a component of an aqueous beverage, i.e. an energy, sports, or soft drink.

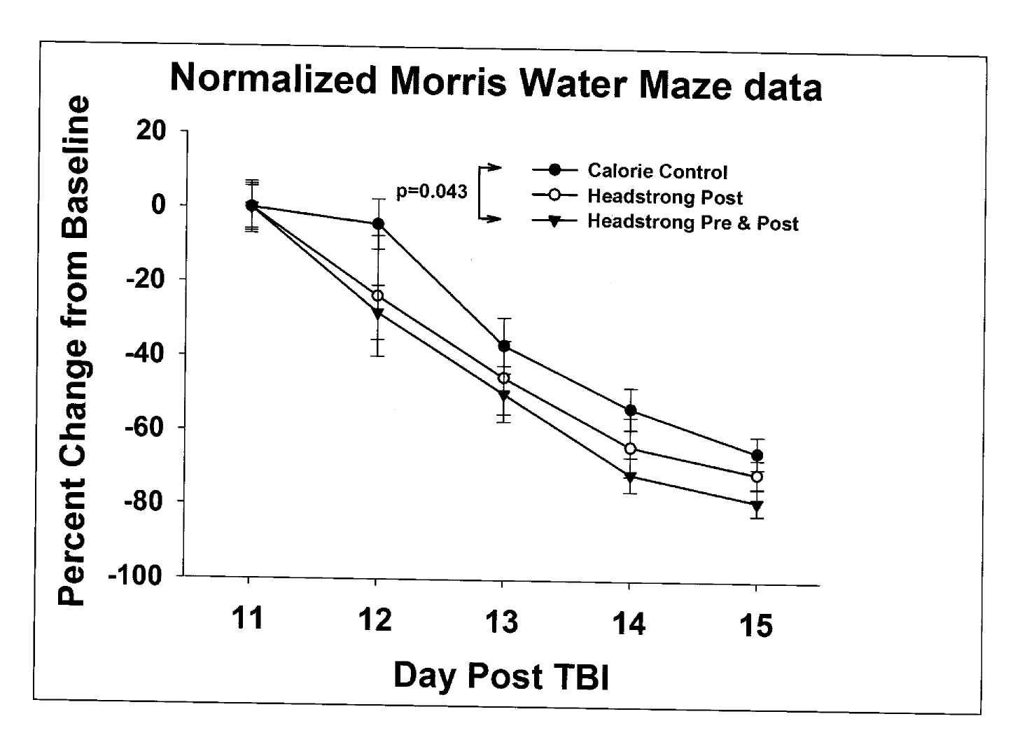

### CROSS-REFERENCE TO RELATED APPLICATIONS

This application is a non-provisional filing claiming priority to U.S. Provisional Patent Application No. 60/984,665, filed on Nov. 1, 2007, which is incorporated herein by reference in its entirety for all purposes.

### FIELD OF THE INVENTION

The present invention relates generally to formulations and methods useful for preconditioning, providing neuroprotection to, and/or enhancing the neural recovery of an individual against trauma, ischemia, neurodegeneration, and their associated cognitive, behavioral and physical impairments.

### BACKGROUND OF THE INVENTION

Neurodegenerative disorders, which are characterized by the dysfunction and death of neurons, have a major impact on society. For instance, one particular neurodegenerative disorder, TBI (traumatic brain injury), poses a major health problem worldwide. TBI, also called acquired brain injury or simply head injury, occurs when a sudden trauma causes damage to the brain. TBI can result when the head suddenly and violently hits an object, or when an object pierces the skull and enters brain tissue. Of the estimated 2 million who sustain a TBI each year in the United States, 75,000 die, 500,000 are hospitalized, and 83,000 survive with permanent disability. The CDC estimates that at least 5.3 million Americans currently require long-term or life-long assistance in day-to-day activities as a result of a TBI.

TBI causes both primary and secondary cell death, with primary tissue damage resulting in immediate cell death and secondary cell death typically taking place over hours or even days. The high incidence of adverse outcomes after a TBI has been attributed in large part to secondary mechanisms of neuronal cell death. Secondary cell death is, however, largely preventable and typically extends over an area beyond the site of initial trauma. The mechanisms for secondary cell death include: cerebral hypoxia, ischemia, glutamate toxicity, free radical production, inflammation, apoptosis and glial invasion. Oftentimes, surgical treatment is needed to remove or repair hematomas or contusions in more severe TBI cases. Effects of TBI include loss of consciousness and/or memory, impaired cognitive abilities, sensory processing, communication, behavior or mental health, and in more serious cases, TBI can leave the injured in a stupor, coma, vegetative state, or persistent vegetative state. In addition to its direct effects, TBI also heightens the risk for developing epilepsy, Parkinson's disease, Alzheimer's disease, dementia pugilistica, and post-traumatic dementia. Turning to the impact in the military arena, Brain Injury Association of America estimates that as of Mar. 24, 2007, a total of 12,274 U.S. service members have sustained a TBI in either Iraq or Afghanistan but projects this number could grow as high as 150,000.

Despite intensive study, conventional neuroprotective strategies for preventing the neuropathological sequelae of TBI have largely failed in translation to clinical treatment. Studies have shown that pretreatment is often required for effective neuroprotection. Thus, there is a substantial need in the art for methods and compositions that precondition, provide neuroprotection to, and/or enhance the neural recovery of individuals against trauma, ischemia, and neurodegeneration. Such methods and compositions are useful for those at risk for TBI, e.g. athletes, military personnel, law enforcement, surgical candidates, children, adolescents, the elderly, or anyone who can benefit from the neuroprotective properties conferred by the present invention, such as those subjected to surgical procedures, or other sources of CNS trauma, and those affected by cerebrovascular diseases, pathological or age-associated neurodegenerative disorders.

The present invention provides compositions having a synergistic effect that is targeted at multiple processes involved in secondary cell death and capable of convenient oral administration as a food or beverage.

### RELEVANT LITERATURE

Loren D J, Seeram N P, Schulman R N, Holtzman D M (2005) “Maternal dietary supplementation with pomegranate juice is neuroprotective in an animal model of neonatal hypoxic-ischemic brain injury,” Pedisir. Res. 57: 858-64.
Wang Y, Chang C, Chou J, Chen H, Deng C, Harvey B K, Cadet J L, and Bickford P C (2005) “Dietary supplementation with blueberries, spinach, or spirulina reduces ischemic brain damage.” Exp. Neurol. 193: 75-84.
Wu A, Ying Z, Gomez-Pinilla F (2006) “Dietary curcumin counteracts the outcome of traumatic brain injury on oxidative stress, synaptic plasticity, and cognition.” Exp. Neurol 197: 309-17.
Wu A, Ying Z, Gomez-Pinilla F (2004) “Dietary omega-3 fatty acids normalize BDNF levels, reduce oxidative damage, and counteract learning disability after traumatic brain injury in rats.” J. Neurotrauma 21: 1457-67.
Temkin N R, Anderson G D, Wirn H R, Ellenbogen R G, Britz G W, schuster J, Lucas T, Newell D W, Mansfield P N, Machamer J E, Barber J, Dikmen S S (2007) “Magnesium sulfate for neuroprotection after traumatic brain injury: a randomized controlled trial.” Lancet Neurol. 6: 29-38.

### SUMMARY OF THE INVENTION

The subject invention provides a composition comprising pomegranate extract, blueberry extract, and curcumin. In some embodiments, the composition also includes magnesium citrate and/or 3 fatty acids. The present invention also provides a beverage useful for providing neuroprotection in an individual. Components of the beverage, which can be formulated as a sports drink, energy drink, or soft drink, includes water, pomegranate extract blueberry extract, and curcumin. In some embodiments, the beverage further contains one or more of any neuroprotective agents known in the art.

Another aspect of the invention provides methods of conferring neuroprotection to a population of cells in an individual by administering to the individual a composition having pomegranate extract, blueberry extract, and curcumin. In some embodiments, the composition being adminstered also includes magnesium substrate and/or omega 3 fatty acids. In other embodiments, administration is followed by exposure of the individual to CNS trauma, preferably TBI. In still other embodiments, administration is followed by subjecting the individual to ischemic injury. In yet other embodiments, the composition is administered to the individual prior to a surgical procedure. In still other embodiments, the method of the invention involves having the individual undergo a neurological test before, during, or after administration.

The methods and compositions of the present invention have other features and advantages which will be apparent from or are set forth in more detail in the accompanying drawing, which is incorporated herein, and the following Detailed Description of the Invention, which together serve to explain certain principles of the present invention.

### BRIEF DESCRIPTION OF THE DRAWINGS

The FIGURE is a graph of the Morris Water Maze data showing the percent change from baseline for three groups (two experimental and one control) as a function of time (number of days after receiving TBI).

### DEFINITIONS

The term “extract” as used herein, includes tinctures, fluid extracts, solid extracts, tars, and infused oils. The parts of the plants used for preparing such extract(s) include one or more of the root, cortices, leaves, stalks, fruits, seeds, and/or blossoms.

Curcumin includes three major components: curcumin, demethoxycurcumin, and bisdemethoxycurcumin, which are often referred to as “curcuminoids.” As used herein, “curcumin” refers to a substance that includes any one or more of these three major components of commercial curcumin, and any active derivative of these agents. This includes natural and synthetic derivatives of curcumin and curcuminoids, and includes any combination of more than one curcumenoid or derivative of curcumin. Derivatives of curcumin and curcumenoids include those derivatives disclosed in U.S. Patent Application Publication 20020019382, Kumar et al., 2000; Mishra et al., 2002; Dinkova-Kostova, 2002; Ohtsu et al., 2002; Ishida et al., 2002; Syu et al., 1998; Sugiyama et al., 1996; Osawa et al., 1995; Naito et al., 2002; Ruby et al., 1995; Rasmussen et al. 2000; Rao et al., 1984; Mukhopadhyay et al., 1982; Rao et al., 1982; Chun et al., 1999; Chun et al., 2002; and Kumar et al., 2003, each of which is herein specifically incorporated by reference.

“Omega-3 fatty acid,” as used herein, encompasses synthetic or naturally occurring forms having a double bond in the third position from the methyl group.

The term “neuroprotection,” as used herein, refers to the capacity of a neuroprotective agent to maintain or stimulate the capacity of neuronal cells to maintain or recover their neuronal functions even in pathological or harmful conditions

An “individual,” as used herein, refers to an animal having a nervous system, particularly a vertebrate, preferably a mammal, more preferably a human.

The term “neurological test,” as used herein, refers to any test, presently known or unknown, that is useful for ascertaining and/or measuring neurological activity and encompasses anatomically-based testing, functional testing, and biochemical assays. Exemplary neurological tests include, without limitation, CAT scan, MRI, Transcranial Doppler, neurosonography, electroencephalogram (EEG), SPECT scan, PET scan, as well as more qualitative examinations of mental status, cranial nerves, motor system, sensory system, the deep tendon reflexes, coordination, the cerebellum, and gait.

### DETAILED DESCRIPTION OF THE INVENTION

Reference will now be made in detail to various embodiments of the present invention(s), examples of which are illustrated in the accompanying drawing and described below. While the invention(s) will be described in conjunction with exemplary embodiments, it will be understood that present description is not intended to limit the invention(s) to those exemplary embodiments. On the contrary, the invention(s) is/are intended to cover not only the exemplary embodiments, but also various alternatives, modifications, equivalents, and other embodiments, which may be included within the spirit and scope of the invention as defined by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the vector” includes reference to one or more vectors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Overview

In general, the present invention provides compositions and methods for preconditioning, providing neuroprotection to, and/or enhancing the neural recovery of an individual against trauma, ischemia, neurodegeneration, and their associated cognitive, behavioral and physical impairments. In an exemplary embodiment, the compositions of the present invention comprise pomegranate extract, blueberry extract, and curcumin.

For ease of administration, the composition of the invention can be formulated as a nutraceutical beverage for ready consumption by an individual. In some aspects of the invention, the components described herein may be packaged separately for consumption. For example, pomegranate juice, blueberry juice, magnesium citrate, and curcumin may be provided in a separate formulation from the Omega 3 fatty acids, which is provided in capsule form. As the timing of CNS injury or, more particularly, TBI, and other causes of ischemic injury cannot often be predicted with certainty, the beverage or food formulation of the invention would allow for ease of consumption, reduced side effects, and increased tolerance in its propylactic use.

For instance, the composition of the invention can be administered immediately prior to, during, and/or after the individual's exposure to a high-risk environment for CNS injury or TBI or before the performance of a surgical procedure on the individual. The composition will confer benefits not only in neurosurgeries but in any surgical procedure that carries a chance of incidentally affecting the integrity of the individual's neurological system. As is familiar to those of ordinary skill in the art, episodes of cerebrovascular ischemia during general anesthesia are relatively common in spite of the advanced monitoring techniques available today. Complications of carotid endarterectomy during local or regional anesthesia, though less common than in general anesthesia, do nevertheless occur. Furthermore, hypotension or inadequate oxygenation is not infrequent and can wreck havoc on the neurological system.

Other cases wherein CNS injury or TBI could be anticipated involve civilians or military personnel in combat zones, athletes engaged in impact sports such as boxing, wrestling, or the martial arts, etc., the elderly population which may be prone to falls and non-pathological, age-associated neurodegeneration, children, adolescents, etc.

Pre-injury administration of the composition will reduce neuronal cell loss and improve behavioral function. As an additional advantage, components of the present invention are readily abundant and commercially found. The combination of pomegranate extract, blueberry extract, curcumin, and in preferred embodiments, magnesium citrate and/or omega 3 fatty acids produces a synergistic effect that targets mechanisms in secondary cell death. Individually, the pomegranate extract acts to reduce glutamate toxicity whereas blueberry extract is a good source of anthocyanins for reducing ischemic cell death. Curcumin provides a source of curcuminoid anti-inflammatory and antioxidants and omega-3 fatty acids, which comprise docosahexaenoic and eicosapentaenoic acids, increase the level of brain-derived neurotrophic factor (BDNF). The inclusion of magnesium citrate also helps to reduce calcium influx. The composition can be administered alone or in combination with therapeutic agents having neuroprotective activity.

### Methods of the Present Invention

The present invention also encompasses methods for providing certain health benefits, particularly, preconditioning, providing neuroprotection to, and/or enhancing the neural recovery of an individual, comprising systemically (generally, orally) administering to an individual having a nervous system, particularly a vertebrate, preferably a mammal, most preferably a human, successive therapeutically effective doses of the present compositions. Such methods include treating, preventing, and/or inhibiting (collectively referred to herein as treating) one or more of the following: trauma, ischemia, neurodegeneration, and their associated cognitive, behavioral and physical impairments.

In accordance with the methods of the present invention, the composition described herein is administered to a mammal, preferably a human. Preferably, such administration is oral. As used herein, the term “oral administration” (or the like) with respect to the individual (preferably, human) means that the individual ingests or is directed to ingest (preferably, for the purpose of treatment of one or more of the various health problems described herein) one or more components of the present invention/compositions of the present invention. Wherein the individual is directed to ingest one or more of the components of the present invention/compositions, such direction may be that which instructs and/or informs the user that use of the composition may and/or will provide treatment for the particular health problem of concern. For example, such direction may be oral direction (e.g., through oral instruction from, for example, a physician, sales professional or organization, and/or radio or television media (i.e., advertisement) or written direction (e.g., through written direction from, for example, a physician or other medical professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” means through words, pictures, symbols, and/or other visible descriptors.

Administration of the present components of the invention/compositions may be via any systemic method, however, such administration is preferably oral. Exemplary modes of administration include oral, rectal, topical, sublingual, transdermal, intravenous infusion, pulmonary, intramuscular, intracavity, aerosol, aural (e.g., via eardrops), intranasal, inhalation, needleless injection, or subcutaneous delivery. Direct injection could also be preferred for local delivery. For continuous infusion, a PCA device may be employed. Oral or subcutaneous administration may be important for prophylactic or long-term treatment because of the convenience of the patient as well as the dosing schedule. Preferred rectal modes of delivery include administration as a suppository or enema wash. For transdermal administration, an ionopheresis device may be employed to enhance penetration of the active drug through the skin. Such devices and methods useful in ionophoresis current assisted transdermal administration include those described in U.S. Pat. Nos. 4,141,359; 5,499,967; and 6,391,015.

In preferred embodiments, the composition of the present invention is administered orally as a beverage or food product. The food product of the present invention can optionally include one or more flavorings and/or natural or artificial color agents to complement the natural juice flavor utilized.

The product can be made in forms that are alternatives to the beverage, and which may contain less water than the beverage, such as puddings, products resembling yogurt, ice cream, and other frozen confections, novelties and desserts, and in the form of food bars.

As non-limiting examples, the components of the present invention can be used in the production of baked goods in any form, such as mixes, shelf-stable baked goods (including food bars), and frozen baked goods. Applications include, but are not limited to, cakes, brownies, muffins, bar cookies, food bars, wafers, biscuits, pastries, pies, pie crusts, and cookies, including sandwich cookies and chocolate chip cookies, particularly the storage-stable dual-textured cookies described in Hong et al., U.S. Pat. No. 4,455,333. The baked goods can contain fruit, cream, or other fillings. Other baked good uses include breads and rolls, crackers, pretzels, pancakes, waffles, ice cream cones and cups, yeast-raised baked goods, pizzas and pizza crusts, baked farinaceous snack foods, and other baked salted snacks.

As stated, food bars are a particularly preferred embodiment of the present invention. The components of the present invention can be incorporated into food bars, such as those described in Greenberg et al., U.S. Pat. No. 5,780,039. The foregoing doses of the present components of the present invention may be included in the advantageous food bars according to the present invention.

In addition to their uses in baked goods, the compositions herein can be used alone or in combination with additional fats to make shortening and oil products. The fats can be synthetic or derived from animal or vegetable sources, or combinations of these. Shortening and oil products include, but are not limited to, shortenings, margarines, spreads, butter blends, lards, cooking and frying oils, salad oils, popcorn oils, salad dressings, mayonnaise, and other edible oil products. In a particular embodiment of the present invention, the compositions are selected from margarines, butter, dressings and spreads.

Other uses for the compositions of the present invention include partial or complete replacement fats and/or oils present in peanut butter, frozen desserts such as ice cream and ice cream coatings, whipped toppings, frosting products, processed meat products, including vegetable protein-based meat analog products, sauces, gravies, and dairy products such as milkshakes, milk products, coffee whiteners, and cheese products.

The components of the present invention described herein are also particularly useful in beverage compositions. Such beverage compositions may be “near-water” beverages (slightly flavored water), milks, coffees, teas, colas, fortified beverages (e.g., calcium fortified beverage), and fruit juices.

Preferred beverage compositions of the present invention are those comprising a beverage member selected from the group consisting of tea solids, milk solids, fruit flavors, botanical flavors, and mixtures thereof. The beverage compositions herein include beverages containing tea solids, and beverage products comprising fruit solids, tea solids, or mixtures thereof. Particularly preferred beverage products comprise both fruit juice and water. Other particularly preferred beverage products comprise both tea solids and water. In another preferred embodiment, “near water” (lightly flavored water) is utilized.

Alternative forms of the present invention can also be prepared by methods known in the art, and may contain, in addition to the components described above, agents to thicken the product, usually to a concentration of 2% by weight, or less. Such agents can include, for example, hydrocolloids such as gelatin, or such as the polysaccharides gum arabic, guar gum carboxymethylcellulose, xanthan, carrageenan, agar, alginates, or pectins. Frozen products are cooled in an ice cream freezer.

Product stability and mouth feel can be improved by using one or more stabilizers. Various food stabilizers can be employed in the present invention and include hydrophilic colloidal stabilizers known as gum arabic, pectins, gelatin, and xanthan as well as the anionic polymers derived from cellulose such as carboxymethyl cellulose. These stabilizers are water soluble and tolerate a low pH.

### Beverage Emulsions

Dilute juice beverages of the present invention may optionally, but preferably, comprise from about 0.2% to about 5%, preferably from about 0.5% to about 3%, and most preferably from about 0.8% to about 2%, of a beverage emulsion. This beverage emulsion can be either a cloud emulsion or a flavor emulsion.

For cloud emulsions, the clouding agent can comprise one or more fats or oils stabilized as an oil-in-water emulsion using a suitable food grade emulsifier. Any of a variety of fats or oils may be employed as the clouding agent, provided that the fat or oil is suitable for use in foods and/or beverages. Preferred are those fats and oils that have been refined, bleached and deodorized to remove off-flavors. Especially suitable for use as clouding agents are those fats that are organoleptically neutral. These include fats from the following sources: vegetable fats such as soybean, corn, safflower, sunflower, cottonseed, canola, and rapeseed; nut fats such as coconut, palm, and palm kernel; and synthetic fats. See e.g., Kupper et al., U.S. Pat. No. 4,705,691, issued Nov. 10, 1987, for suitable fat or oil clouding agents.

Any suitable food grade emulsifier can be used that can stabilize the fat or oil clouding agent as an oil-in-water emulsion. Suitable emulsifiers include gum acacia, modified food starches (e.g., alkenylsuccinate modified food starches), anionic polymers derived from cellulose (e.g., carboxymethylcellulose), gum ghatti, modified gum ghatti, xanthan gum, tragacanth gum, guar gum, locust bean gum, pectin, and mixtures thereof. See e.g., Kupper et al., U.S. Pat. No. 4,705,691, issued Nov. 10, 1987. Modified starches treated to contain hydrophobic as well as hydrophilic groups, such as those described in Caldwell et al., U.S. Pat. No. 2,661,349, are preferred emulsifiers for use as herein. Octenyl succinate (OCS) modified starches such as those described in Marotta et al., U.S. Pat. No. 3,455,838 and Barndt et al., U.S. Pat. No. 4,460,617 are especially preferred emulsifiers.

The clouding agent can be combined with a weighting agent to provide a beverage opacifier that imparts a total or partial opaque effect to the beverage without separating out and rising to the top. The beverage opacifier provides the appearance to the consumer of a juice-containing beverage. Any suitable weighting oil can be employed in the beverage opacifier. Typical weighting oils include brominated vegetable oil, glycerol ester of wood rosin (ester gum), sucrose acetate isobutyrate (SAIB) and other sucrose esters, gum damar, colophony, gum elemi, or others known to those skilled in the art. Other suitable weighting agents include brominated liquid polyol polyesters which are nondigestible. See e.g., Brand et al., U.S. Pat. No. 4,705,690, issued Nov. 10, 1987.

The cloud/opacifier emulsion is prepared by mixing the clouding agent with the weighting agent (for opacifier emulsions), the emulsifier and water. The emulsion typically contains from about 0.1% to about 25% clouding agent, from about 1% to about 20% weighting oil agent (in the case of opacifier emulsions), from about 1% to about 30% emulsifiers, and from about 25% to about 97.9% water (or quantum satis).

The particle size of the water-insoluble components of the emulsion is reduced by employing a suitable apparatus known in the art. Because the ability of emulsifying agents to hold oil in suspension is proportional to particle size, emulsions of particles with diameters of about 0.1 to about 3.0 microns are suitable. Preferably, the particles are about 2.0 microns or less in diameter. Most preferred is an emulsion in which substantially all the particles are 1.0 microns or less in diameter. The particle size is reduced by passing the mixture through an homogenizer, colloid mill or turbine-type agitator. Usually one or two passes is sufficient. See e.g., Kupper et al., U.S. Pat. No. 4,705,691, issued Nov. 10, 1987.

Flavor emulsions useful in beverage products of the present invention comprise one or more suitable flavor oils, extracts, oleoresins, essential oils and the like, known in the art for use as flavorants in beverages. This component can also comprise flavor concentrates such as those derived from concentration of natural products such as fruits. Terpeneless citrus oils and essences can also be used herein. Examples of suitable flavors include, for example, fruit flavors such as orange, lemon, lime and the like, cola flavors, tea flavors, coffee flavors, chocolate flavors, dairy flavors. These flavors can be derived from natural sources such as essential oils and extracts, or can be synthetically prepared. The flavor emulsion typically comprises a blend of various flavors and can be employed in the form of an emulsion, alcoholic extract, or spray dried. The flavor emulsion can also include clouding agents, with or without weighting agents, as previously described. See e.g., Kupper et al., U.S. Pat. No. 4,705,691, issued Nov. 10, 1987.

Flavor emulsions are typically prepared in the same manner as cloud/opacifier emulsions by mixing one or more flavoring oils (from about 0.001% to about 20%) with an emulsifying agent (from about 1% to about 30%) and water. (The oil clouding agents can also be present). Emulsions of particles with diameters of from about 0.1 to about 3.0 microns are suitable. Preferably, the particles are about 2.0 microns or less in diameter. Most preferably, the particles are about 1.0 microns or less in diameter. The emulsifying agent coats the particularized flavor oil to aid in preventing coalescence and in maintaining an appropriate dispersion. The viscosity and specific gravity of the flavor emulsion are regulated to be compatible with the finished beverage. See e.g., Kupper et al., U.S. Pat. No. 4,705,691, issued Nov. 10, 1987.

### Surfactants

The compositions, according to the invention, may optionally comprise one or more surfactants. Examples of suitable surfactants include, but are not limited to, sorbitan monostearate, which is marketed under the trade name Span 60; sorbitan tristearate which is marketed under the trade name Span 65; POE(20) sorbitan mono stearate which is marketed under the trade name Tween 60; POE(20) sorbitan tristearate which is marketed under the trade name Tween 65; POE(20) sorbitan monooleate which is marketed under the trade name Tween 80; polyoxyethylene (20) monolaurate which is marketed under the trade name Tween 20; polyoxyethylene (8) stearate which is marketed under the trade name Myrj 45; and polyoxyethylene (40) stearate which is marketed under the trade name Myrj 52; all of which are sold by ICI Surfactants of Wilmington, Del. U.S.A. Additional examples of suitable surfactants include acid esters of monoglycerides, which are marketed under the trade name Panodan by Danisco Ingredients of New Century, Kans. U.S.A., and glycerol esters, which are marketed under the trade names Caprol PGE 860 and Caprol 3 GO by Abitec Corp. of Janesville, Wis. U.S.A.

### Flavor Agents

The compositions herein may optionally, but preferably, comprise one or more flavor agents. Preferably, such flavor agents are included in the beverage compositions and are typically selected from fruit juice, tea solids, milk solids, fruit flavors, botanical flavors, and mixtures thereof. Wherein fruit juice is included, the beverages of the present invention can comprise from about 0.1% to about 40%, preferably from about 11% to about 20%, more preferably from about 2% to about 10%, and most preferably from about 3% to about 6%, fruit juice. (As measured herein, the weight percentage of fruit juice is based on a single strength 2.degree. to 16.degree. Brix fruit juice). The fruit juice can be incorporated into the beverage as a puree, comminute, or as a single strength or concentrated juice. Especially preferred is incorporation of the fruit juice as a concentrate with a solids content (primarily as sugar solids) of from about 20.degree. to about 80.degree. Brix.

The fruit juice can be any citrus juice, non-citrus juice, or mixture thereof, which are known for use in dilute juice beverages. The juice can be derived from, for example, apple, cranberry, pear, peach, plum, apricot, nectarine, grape, cherry, currant, raspberry, gooseberry, elderberry, blackberry, blueberry, strawberry, lemon, lime, mandarin, orange, grapefruit, cupuacu, potato, tomato, lettuce, celery, spinach, cabbage, watercress, dandelion, rhubarb, carrot, beet, cucumber, pineapple, coconut, pomegranate, kiwi, mango, papaya, banana, watermelon, passion fruit, tangerine, and cantaloupe. Preferred juices are derived from apple, pear, lemon, lime, mandarin, grapefruit, cranberry, orange, strawberry, tangerine, grape, kiwi, pineapple, passion fruit, mango, guava, raspberry and cherry. Citrus juices, preferably grapefruit, orange, lemon, lime, and mandarin juices, as well as juices derived from mango, apple, passion fruit, and guava, as well as mixtures of these juices are most preferred.

Fruit flavors may also be utilized. As described above with respect to flavor emulsions, fruit flavors may be derived from natural sources such as essential oil and extracts, or can be synthetically prepared. Fruit flavors may be derived from fruits through processing, particularly concentrating. Wherein fruit juices are concentrated or evaporated, the water which is removed or the condensate contains volatile substances which comprise the flavor of the fruit. Often, such flavor is added to a juice concentrate to enhance the flavor thereof. The condensate may also be used to flavor “near waters” (lightly flavored water).

Botanical flavors may also be utilized. As used herein, the term “botanical flavor” refers to a flavor derived from parts of a plant other than the fruit; i.e., derived from nuts, bark, roots, and/or leaves. Also included within the term “botanical flavor” are synthetically prepared flavors made to simulate botanical flavors derived from natural sources. Botanical flavors can be derived from natural sources such as essential oils and extracts, or can be synthetically prepared. Suitable botanical flavors include jamaica, kola, marigold, chrysanthemum, chamomile, ginger, valerian, yohimbe, hops, eriodictyon, ginseng, bilberry, rice, red wine, mango, peony, lemon balm, nut gall, oak chip, lavender, walnut, gentiam, luo han guo, cinnamon, angelica, aloe, agrimony, yarrow and mixtures thereof.

Tannic acid or other similar acids can be used to provide an astringent taste to the beverage. From about 0.001% to about 10% tannic acid is used. Other flavor enhancers, as well as flavorants such as chocolate and vanilla can also be used.

Wherein tea solids are included, the beverages of the present invention can comprise from about 0.01% to about 1.2%, preferably from about 0.05% to about 0.8%, by weight of the beverage product, of tea solids. The term “tea solids” as used herein means solids extracted from tea materials including those materials obtained from the genus Camellia including C. sinensis and C. assaimica, for instance, freshly gathered tea leaves, fresh green tea leaves that are dried immediately after gathering, fresh green tea leaves that have been heat treated before drying to inactivate any enzymes present, unfermented tea, instant green tea, and partially fermented tea leaves. Green tea materials are tea leaves, tea plant stems, and other plant materials that are related and which have not undergone substantial fermentation to create black teas. Members of the genus Phyllanthus, Catechu gambir and Uncaria family of tea plants can also be used. Mixtures of unfermented and partially fermented teas can be used.

Tea solids for use in beverages of the present invention can be obtained by known and conventional tea solid extraction methods. A particularly preferred source of green tea solids can be obtained by the method described in Ekanayake et al., U.S. application Ser. No. 08/606,907, filed Feb. 26, 1996. Tea solids so obtained will typically comprise caffeine, theobromine, proteins, amino acids, minerals and carbohydrates. Suitable beverages containing tea solids can be formulated according to Tsai et al., U.S. Pat. No. 4,946,701, issued Aug. 7, 1990. See also, Ekanayake et al., U.S. Pat. No. 5,427,806, issued Jun. 26, 1995, for a suitable sources of green tea solids for use in the present invention.

Beverages according to the present invention may also comprise milk solids. These milk solids can be derived from various sources including whole milk, skim milk, condensed milk, and dried milk powder. As used herein, the term “milk” will be used to describe an aqueous dispersion of milk solids, such as fluid (whole or skim milk) or non-fat dry milk or condensed milk diluted with water. The amount of milk included typically ranges from about 5% to about 99.8%, preferably from about 5% to about 75%, more preferably from about 5% to about 40%, and most preferably from about 5% to about 15%. The amount of non-fat milk solids correlating to these levels of milk solids is in the range of from about 0.5% to about 8.2%, from about 0.5% to about 6.2%, from about 0.5% to about 3.3%, and from about 0.5% to 1.2% of the beverage, respectively.

### Thickeners and Bulking Agents

Food and beverage compositions according to the present invention can further comprise one or more thickeners or bulking agents, including xanthan gum, carboxymethylcellulose, carboxyethylcellulose, hydroxypropylcellulose, methylcellulose, microcrystalline cellulose, starches, dextrins, fermented whey, tofu, maltodextrins, polyols, including sugar alcohols (e.g., sorbitol and mannitol), carbohydrates (e.g., lactose), propylene glycol alginate, gellan gum, guar gum, pectin, tragacanth gum, gum acacia, locust bean gum, gum arabic, gelatin, as well as mixtures of these thickeners. These thickeners and bulking agents are typically included in the compositions of the present invention at levels up to about 0.1%, depending on the particular thickener involved and the viscosity effects desired.

### Sweeteners

The food and beverage compositions of the present invention can, and typically will, contain an effective amount of one or more sweeteners, including carbohydrate sweeteners and natural and/or artificial no/low calorie sweeteners. The amount of the sweetener used in the compositions of the present invention typically depends upon the particular sweetener used and the sweetness intensity desired. For no/low calorie sweeteners, this amount varies depending upon the sweetness intensity of the particular sweetener.

The compositions of the present invention can be sweetened with any of the carbohydrate sweeteners, preferably monosaccharides and/or disaccharides. Sweetened compositions, particularly beverages, will typically comprise from about 0.1% to about 20%, most preferably from about 6 to about 14%, sweetener. These sweeteners can be incorporated into the compositions in solid or liquid form but are typically, and preferably, incorporated as a syrup, most preferably as a concentrated syrup such as high fructose corn syrup. For purposes of preparing beverages of the present invention, these sugar sweeteners can be provided to some extent by other components of the beverage such as, for example, the fruit juice component and/or flavors.

Preferred sugar sweeteners for use in compositions of the present invention are sucrose, fructose, glucose, and mixtures thereof. Fructose can be obtained or provided as liquid fructose, high fructose corn syrup, dry fructose or fructose syrup, but is preferably provided as high fructose corn syrup. High fructose corn syrup (HFCS) is commercially available as HFCS-42, HFCS-55 and HFCS-90, which comprise 42%, 55% and 90%, respectively, by weight of the sugar solids therein, as fructose. Other naturally occurring sweeteners or their purified extracts, such as glycyrrhizin, the protein sweetener thaumatin, the juice of Luo Han Guo disclosed in, for example, Fischer et al., U.S. Pat. No. 5,433,965, issued Jul. 18, 1995, and the like can also be used in the compositions of the present invention.

Suitable no/low calorie sweeteners include saccharin, cyclamates, L-aspartyl-L-phenylalanine lower alkyl ester sweeteners (e.g., aspartame); L-aspartyl-D-alanine amides disclosed in Brennan et al., U.S. Pat. No. 4,411,925; L-aspartyl-D-serine amides disclosed in Brennan et al., U.S. Pat. No. 4,399,163; L-aspartyl-L-1-hydroxymethylalkaneamide sweeteners disclosed in Brand, U.S. Pat. No. 4,338,346; L-aspartyl-1-hydroxyethyalkaneamide sweeteners disclosed in Rizzi, U.S. Pat. No. 4,423,029; L-aspartyl-D-phenylglycine ester and amide sweeteners disclosed in Janusz, European Patent Application 168,112, published Jan. 15, 1986; N—[N-3,3-dimethylbutyl)-L-.quadrature.-aspartyl]-L-phenylalanin-e 1-methyl ester sweeteners disclosed in Gerlat et al., WO 99/30576, assigned to The Nutrasweet Co., published Jun. 24, 1999; alltame, thaumatin; dihydrochalcones; cyclamates; steviosides; glycyrrhizins, synthetic alkoxy aromatics, such as Dulcin and P-4000; sucrolose; suosan; miraculin; monellin; sorbitol, xylitol; talin; cyclohexylsulfamates; substituted imidazolines; synthetic sulfamic acids such as acesulfame, acesulfame-K and n-substituted sulfamic acids; oximes such as perilartine; rebaudioside-A; peptides such as aspartyl malonates and succanilic acids; dipeptides; amino acid based sweeteners such as gem-diaminoalkanes, meta-aminobenzoic acid, L-aminodicarboxylic acid alkanes, and amides of certain alpha-aminodicarboxylic acids and gem-diamines; and 3-hydroxy-4-alkyloxyphenyl aliphatic carboxylates or heterocyclic aromatic carboxylates; and the like and mixtures thereof. A particularly preferred low calorie sweetener is aspartame.

### Coloring Agent

Small amounts of coloring agents may be utilized in the compositions of the present invention. FD&C dyes (e.g., yellow #5, blue #2, red #40) and/or FD&C lakes are preferably used. By adding the lakes to the other powdered ingredients, all the particles, in particular the colored iron compound, are completely and uniformly colored and a uniformly colored composition is attained. Preferred lake dyes which may be used in the present invention are the FDA-approved Lake, such as Lake red #40, yellow #6, blue #1, and the like. Additionally, a mixture of FD&C dyes or a FD&C lake dye in combination with other conventional food and food colorants may be used. Riboflavin and .quadrature.-carotene may also be used. The exact amount of coloring agent used will vary, depending on the agents used and the intensity desired in the finished product. The amount can be readily determined by one skilled in the art. Generally, if utilized, the coloring agent should be present at a level of from about 0.0001% to about 0.5%, preferably from about 0.001% to about 0.1%, and most preferably from about 0.004% to about 0.1%, by weight of the composition.

### Nutrients

The compositions herein (particularly the food and beverage compositions) can be fortified with one or more nutrients, especially one or more vitamins, minerals, and/or amino acids. The U.S. Recommended Daily Intake (USRDI) for vitamins and minerals are defined and set forth in the Recommended Daily Dietary Allowance-Food and Nutrition Board, National Academy of Sciences-National Research Council.

Any amino acid may be utilized herein, especially the naturally occurring amino acids. Preferred amino acids for inclusion herein are L-lysine and L-carnitine, particularly L-lysine.

Unless otherwise specified herein, wherein a given mineral is present in the product, the product comprises at least about 1%, preferably at least about 5%, more preferably from about 10% to about 200%, even more preferably from about 40% to about 150%, and most preferably from about 60% to about 125% of the USRDI of such mineral. Unless otherwise specified herein, wherein a given vitamin is present in the product, the product comprises at least about 1%, preferably at least about 5%, more preferably from about 10% to about 200%, even more preferably from about 20% to about 150%, and most preferably from about 25% to about 120% of the USRDI of such vitamin.

Non-limiting examples of such vitamins and minerals include iron, zinc, copper, calcium, phosphorous, niacin, thiamin, folic acid, pantothenic acid, iodine, vitamin A, vitamin C, vitamin B₂, vitamin B₃, vitamin B₆, vitamin B₁₂, vitamin D, vitamin E, and vitamin K. Preferably, wherein a vitamin or mineral is utilized the vitamin or mineral is selected from iron, zinc, calcium, niacin, thiamin, folic acid, iodine, vitamin A, vitamin C, vitamin B₆, vitamin B₁₂, vitamin D, and vitamin E. A particularly preferred mineral for use herein is calcium.

Commercially available vitamin A sources may also be included in the present compositions. Vitamin A can be provided, for example, as vitamin A palmitate (retinol palmitate) and/or as beta-carotene. The vitamin A may be in the form of, for example, an oil, beadlets or encapsulated. As used herein, “vitamin A” includes, but is not limited to, vitamin A, .beta.-carotene, retinol palmitate, and retinol acetate. Wherein vitamin A is present in the compositions herein, the product comprises at least about 1%, preferably at least about 5%, more preferably from about 10% to about 200%, even more preferably from about 15% to about 150%, and most preferably from about 20% to about 120% of the USRDI of such vitamin. Wherein vitamin A is present in the products herein, it is especially preferred to include about 25% of the USRDI of vitamin A. The quantity of vitamin A to be added is dependent on processing conditions and the amount of vitamin A deliver desired after storage. Preferably, wherein vitamin A is included within the present compositions, the products comprise from about 0.0001% to about 0.2%, more preferably from about 0.0002% to about 0.12%, also preferably from about 0.0003% to about 0.1%, even more preferably from about 0.0005% to about 0.08%, and most preferably from about 0.001% to about 0.06% of vitamin A, by weight of the composition.

Commercially available sources of vitamin B₂ (also known as riboflavin) may be utilized in the present compositions. Wherein vitamin B₂ is present in the compositions herein, the product comprises at least about 1%, preferably at least about 5%, more preferably from about 5% to about 200%, even more preferably from about 10% to about 150%, and most preferably from about 10% to about 120% of the USRDI of such vitamin. Wherein vitamin B₂ is present in the compositions herein, it is especially preferred to include from about 15% to about 35% of the USRDI of vitamin B₂.

Commercially available sources of vitamin C can be used herein. Encapsulated ascorbic acid and edible salts of ascorbic acid can also be used. Wherein vitamin C is present in the products herein, the product comprises at least about 1%, preferably at least about 5%, more preferably from about 10% to about 200%, even more preferably from about 20% to about 150%, and most preferably from about 25% to about 120% of the USRDI of such vitamin. Wherein vitamin C is present in the compositions herein, it is especially preferred to include about 100% of the USRDI of vitamin C. The quantity of vitamin C to be added is dependent on processing conditions and the amount of vitamin C deliver desired after storage. Preferably, wherein vitamin C is included within the present compositions, the compositions comprise from about 0.005% to about 0.2%, more preferably from about 0.01% to about 0.12%, also preferably from about 0.02% to about 0.1%, even more preferably from about 0.02% to about 0.08%, and most preferably from about 0.03% to about 0.06% of vitamin C, by weight of the composition.

Commercial sources of iodine, preferably as an encapsulated iodine may be utilized herein. Other sources of iodine include iodine-containing salts, e.g., sodium iodide, potassium iodide, potassium iodate, sodium iodate, or mixtures thereof. These salts may be encapsulated.

Nutritionally supplemental amounts of other vitamins which may be incorporated herein include, but are not limited to, vitamins B.sub.6 and B.sub.12, folic acid, niacin, pantothenic acid, folic acid, vitamin D, and vitamin E. Wherein the composition comprises one of these vitamins, the product preferably comprises at least 5%, preferably at least 25%, and most preferably at least 35% of the USRDI for such vitamin.

Minerals which may optionally be included in the composition herein are, for example, magnesium, zinc, iodine, iron, and copper. Any soluble salt of these minerals suitable for inclusion edible products can be used, for example, magnesium citrate, magnesium gluconate, magnesium sulfate, zinc chloride, zinc sulfate, potassium iodide, copper sulfate, copper gluconate, and copper citrate.

Calcium is a particularly preferred mineral for use in the present invention. Preferred sources of calcium include, for example, amino acid chelated calcium, calcium carbonate, calcium oxide, calcium hydroxide, calcium sulfate, calcium chloride, calcium phosphate, calcium hydrogen phosphate, calcium dihydrogen phosphate, calcium citrate, calcium malate, calcium titrate, calcium gluconate, calcium realate, calcium tantrate, and calcium lactate, and in particular calcium citrate-malate. The form of calcium citrate-malate is described in, e.g., Mehansho et al., U.S. Pat. No. 5,670,344, issued Sep. 23, 1997; Diehl et al., U.S. Pat. No. 5,612,026, issued Mar. 18, 1997; Andon et al., U.S. Pat. No. 5,571,441, issued Nov. 5, 1996; Meyer et al., U.S. Pat. No. 5,474,793, issued Dec. 12, 1995; Andon et al., U.S. Pat. No. 5,468,506, issued Nov. 21, 1995; Burkes et al., U.S. Pat. No. 5,445,837, issued Aug. 29, 1995; Dake et al., U.S. Pat. No. 5,424,082, issued Jun. 13, 1995; Burkes et al., U.S. Pat. No. 5,422,128, issued Jun. 6, 1995; Burkes et al., U.S. Pat. No. 5,401,524, issued Mar. 28, 1995; Zuniga et al., U.S. Pat. No. 5,389,387, issued Feb. 14, 1995; Jacobs, U.S. Pat. No. 5,314,919, issued May 24, 1994; Saltman et al., U.S. Pat. No. 5,232,709, issued Aug. 3, 1993; Camden et al., U.S. Pat. No. 5,225,221, issued Jul. 6, 1993; Fox et al., U.S. Pat. No. 5,215,769, issued Jun. 1, 1993; Fox et al., U.S. Pat. No. 5,186,965, issued Feb. 16, 1993; Saltman et al., U.S. Pat. No. 5,151,274, issued Sep. 29, 1992; Kochanowski, U.S. Pat. No. 5,128,374, issued Jul. 7, 1992; Mehansho et al., U.S. Pat. No. 5,118,513, issued Jun. 2, 1992; Andon et al., U.S. Pat. No. 5,108,761, issued Apr. 28, 1992; Mehansho et al., U.S. Pat. No. 4,994,283, issued Feb. 19, 1991; Nakel et al., U.S. Pat. No. 4,786,510, issued Nov. 22, 1988; and Nakel et al., U.S. Pat. No. 4,737,375, issued Apr. 12, 1988. Preferred compositions of the present invention will comprise from about 0.01% to about 0.5%, more preferably from about 0.03% to about 0.2%, even more preferably from about 0.05% to about 0.15%, and most preferably from about 0.1% to about 0.15% of calcium, by weight of the composition.

Iron may also be utilized in the compositions of the present invention. Acceptable forms of iron are well-known in the art. The amount of iron compound incorporated into the composition will vary widely depending upon the level of supplementation desired in the final product and the targeted consumer. Iron fortified compositions of the present invention typically contain from about 5% to about 100%, preferably from about 15% to about 50%, and most preferably about 20% to about 40% of the USRDI for iron.

Ferrous iron is typically better utilized by the body than ferric iron. Highly bioavailable ferrous salts that can be used in the ingestible compositions of the present invention are ferrous sulfate, ferrous fumarate, ferrous succinate, ferrous gluconate, ferrous lactate, ferrous tartarate, ferrous citrate, ferrous amino acid chelates, as well as mixtures of these ferrous salts. While ferrous iron is typically more bioavailable, certain ferric salts can also provide highly bioavailable sources of iron. Highly bioavailable ferric salts that can be used in the food or beverage compositions of the present invention are ferric saccharate, ferric ammonium citrate, ferric citrate, ferric sulfate, as well as mixtures of these ferric salts. Combinations or mixtures of highly bioavailable ferrous and ferric salts can be used in these edible mixes and ready-to-serve beverages. The preferred sources of highly bioavailable iron are ferrous fumarate and ferrous amino acid chelates.

Ferrous amino acid chelates particularly suitable as highly bioavailable iron sources for use in the present invention are those having a ligand to metal ratio of at least 2:1. For example, suitable ferrous amino acid chelates having a ligand to metal mole ratio of two are those of formula:

Fe(L)₂

where L is an alpha amino acid, dipeptide, tripeptide, or quadrapeptide ligand. Thus, L can be any ligand which is a naturally occurring alpha amino acid selected from alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, and valine; or dipeptides, tripeptides, or quadrapeptides formed by any combination of these alpha amino acids. See e.g., Ashmead et al., U.S. Pat. No. 4,863,898, issued Sep. 5, 1989; Ashmead, U.S. Pat. No. 4,830,716, issued May 16, 1989; and Ashmead, U.S. Pat. No. 4,599,152, issued Jul. 8, 1986, all of which are incorporated by reference. Particularly preferred ferrous amino acid chelates are those where the reacting ligands are glycine, lysine, and leucine. Most preferred is the ferrous amino acid chelate sold under the mark Ferrochel® (Albion Laboratories, Salt Lake City, Utah) wherein the ligand is glycine.

In addition to these highly bioavailable ferrous and ferric salts, other sources of bioavailable iron can be included in the food and beverage compositions of the present invention. Other sources of iron particularly suitable for fortifying products of the present invention included certain iron-sugar-carboxylate complexes. In these iron-sugar-carboxylate complexes, the carboxylate provides the counterion for the ferrous (preferred) or ferric iron. The overall synthesis of these iron-sugar-carboxylate complexes involves the formation of a calcium-sugar moiety in aqueous media (for example, by reacting calcium hydroxide with a sugar, reacting the iron source (such as ferrous ammonium sulfate) with the calcium-sugar moiety in aqueous media to provide an iron-sugar moiety, and neutralizing the reaction system with a carboxylic acid (the “carboxylate counterion”) to provide the desired iron-sugar-carboxylate complex. Sugars that can be used to prepare the calcium-sugar moiety include any of the ingestible saccharidic materials, and mixtures thereof, such as glucose, sucrose and fructose, mannose, galactose, lactose, maltose, and the like, with sucrose and fructose being the more preferred. The carboxylic acid providing the “carboxylate counterion” can be any ingestible carboxylic acid such as citric acid, malic acid tartaric acid, lactic acid, succinic acid, propionic acid, etc., as well as mixtures of these acids.

These iron-sugar-carboxylate complexes can be prepared in the manner described in, e.g., Nakel et al., U.S. Pat. Nos. 4,786,510 and 4,786,518, issued Nov. 22, 1988, both of which are incorporated by reference. These materials are referred to as “complexes”, but they may exist in solution as complicated, highly hydrated, protected colloids; the term “complex” is used for the purpose of simplicity.

Zinc may also be utilized in the compositions of the present invention. Acceptable forms of zinc are well-known in the art. Zinc fortified products of the present invention typically contain from about 5% to about 100%, preferably from about 15% to about 50%, and most preferably about 25% to about 45% of the USRDI for zinc. The zinc components of the present invention which can be used in the present invention can be in any of the commonly used forms such as, e.g., zinc sulfate, zinc chloride, zinc acetate, zinc gluconate, zinc ascorbate, zinc citrate, zinc aspartate, zinc picolinate, amino acid chelated zinc, and zinc oxide. Zinc gluconate and amino acid chelated zinc are particularly preferred.

### Carbonation

Carbon dioxide can be introduced into the water which is mixed with a beverage syrup or into the dilute beverage after dilution to achieve carbonation. The carbonated beverage can be placed into a container, such as a bottle or can, and then sealed. Any conventional carbonation methodology may be utilized to make carbonated beverage products of this invention. The amount of carbon dioxide introduced into the beverage will depend upon the particular flavor system utilized and the amount of carbonation desired.

pH

The compositions of the present invention, particularly the beverage compositions, preferably have a pH of from about 2 to about 8, more preferably from about 2 to about 4.5, and most preferably from about 2.7 to about 4.2. Beverage acidity can be adjusted to and maintained within the requisite range by known and conventional methods, e.g., the use of food grade acid buffers. Typically, beverage acidity within the above recited ranges is a balance between maximum acidity for microbial inhibition and optimum acidity for the desired beverage flavor. Food compositions preferably have a pH of less than about 8.

### Fiber Component

Similarly, food and beverage compositions can be made that combine the present compositions with dietary fibers to achieve the combined benefits of each. By “dietary fiber” is meant complex carbohydrates resistant to digestion by mammalian enzymes, such as the carbohydrates found in plant cell walls and seaweed, and those produced by microbial fermentation. Examples of these complex carbohydrates are brans, celluloses, hemicelluloses, pectins, gums and mucilages, seaweed extract, and biosynthetic gums. Sources of the cellulosic fiber include vegetables, fruits, seeds, cereals, and man-made fibers (for example, by bacterial synthesis). Commercial fibers such as purified plant cellulose, or cellulose flour, can also be used. Naturally occurring fibers include fiber from whole citrus peel, citrus albedo, sugar beets, citrus pulp and vesicle solids, apples, apricots, and watermelon rinds.

These dietary fibers may be in a crude or purified form. The dietary fiber used may be of a single type (e.g., cellulose), a composite dietary fiber (e.g., citrus albedo fiber containing cellulose and pectin), or some combination of fibers (e.g., cellulose and a gum). The fibers can be processed by methods known to the art.

Primarily due to the present compositions, the foods and beverages herein can help reduce serum cholesterol and the risk of developing cardiovascular and atherosclerotic diseases. Additionally, the present compositions have acceptable organoleptic properties, particularly flavor and texture.

Dietary foods can be made with the compositions to meet special dietary needs, for example, of persons who are obese, diabetic, or hypercholesterolemic. The present compositions can be a major part of a low-fat, low-calorie, low-cholesterol diet, or may supplement a normal diet, and they can be used alone or in combination with drug therapy, nutritional therapy, or other therapy. Combinations of food or beverage products made with the compositions can be used as part of a total dietary management regimen, based on one or more of these products, containing the compositions alone or in combination with one or more of the above-mentioned ingredients, to provide one or more of the above-mentioned benefits.

### Preservatives

Preservatives may optionally be added to the product to extend product shelf life. If desired, preservatives such as sorbate, benzoate, polyphosphate, or calcium disodium EDTA can be used.

Preferably, wherein a preservative is utilized herein, one or more sorbate or benzoate preservatives (or mixtures thereof) are utilized. Sorbate and benzoate preservatives suitable for use in the present invention include sorbic acid, benzoic acid, and salts thereof, including (but not limited to) calcium sorbate, sodium sorbate, potassium sorbate, calcium benzoate, sodium benzoate, potassium benzoate, and mixtures thereof. Sorbate preservatives are particularly preferred. Potassium sorbate is particularly preferred for use in the present invention.

Wherein a composition comprises a preservative, the preservative is preferably included at levels from about 0.0005% to about 0.5%, more preferably from about 0.001% to about 0.4% of the preservative, still more preferably from about 0.001% to about 0.1%, even more preferably from about 0.001% to about 0.05%, and most preferably from about 0.003% to about 0.03% of the preservative, by weight of the composition. Wherein the composition comprises a mixture of one or more preservatives, the total concentration of such preservatives is preferably maintained within these ranges.

### Alternative Routes of Administration

In some embodiments of the invention, the composition is administered as a pharmaceutical preparation which may be presented in unit dosage form and may be prepared by any of the methods well known in the art of pharmacy. All methods include the step of bringing the components of the present invention of the invention into association with a carrier which constitutes one or more accessory ingredients. In general, the compositions are prepared by uniformly and intimately bringing the components of the present invention of the invention into association with a liquid carrier, a finely divided solid carrier, or both, and then, if necessary, shaping the product.

When administered, the pharmaceutical preparations of the invention are applied in pharmaceutically acceptable compositions. Such preparations may routinely contain salts, buffering agents, preservatives, compatible carriers, lubricants and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulphuric, nitric, phosphoric, maleic, acetic, salicylic, p-toluenesulfonic, tartaric, citric, methanesulfonic, formic, succinic, naphthalene-2-sulfonic, pamoic, 3-hydroxy-2-naphthalenecarboxylic, and benzene sulfonic.

The pharmaceutical preparations of the present invention may include or be diluted into a pharmaceutically-acceptable carrier. The term “pharmaceutically-acceptable carrier” as used herein means one or more compatible solid or liquid filler, diluents or encapsulating substances which are suitable for administration to a human or other mammal such as a dog, cat, horse, cow, sheep, or goat. The term “carrier” denotes an organic or inorganic ingredient, natural or synthetic, with which the active ingredient is combined to facilitate the application. The carriers are capable of being commingled with the preparations of the present invention, and with each other, in a manner such that there is no interaction which would substantially impair the desired pharmaceutical efficacy or stability. Carrier formulations suitable for oral administration, for suppositories, and for parenteral administration, etc., can be found in Remington's Pharmaceutical Sciences, Mack Publishing Company, Easton, Pa.

Aqueous formulations may include one or more of a chelating agent, a buffering agent, an anti-oxidant, an isotonicity agent, and a preservative. In the case of quaternary amine derivatives of noroxymorphone, a chelating agent can be added and pH can be adjusted to between 3.0 and 3.5. Preferred such formulations that are stable to autoclaving and long term storage are described in co-pending application Ser. No. 60/461,611, filed on the same date hereof, entitled “Pharmaceutical Formulation”, the disclosure of which is incorporated herein by reference.

Chelating agents include: ethylenediaminetetraacetic acid (EDTA) and derivatives thereof, citric acid and derivatives thereof, niacinamide and derivatives thereof, sodium desoxycholate and derivatives thereof.

Buffering agents include: citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate and benzoic acid, and combinations thereof.

Antioxidants include: those selected from the group consisting of an ascorbic acid derivative, butylated hydroxy anisole, butylated hydroxy toluene, alkyl gallate, sodium meta-bisulfite, sodium bisulfite, sodium dithionite, sodium thioglycollate, sodium formaldehyde sulfoxylate, tocopheral and derivatives thereof, monothioglycerol, and sodium sulfite. The preferred antioxidant is monothioglycerol.

Isotonicity agents include: those selected from the group consisting of sodium chloride, mannitol, lactose, dextrose, glycerol, and sorbitol.

Any of the active agents (i.e., ingredients) may be provided in particles. Particles as used herein means nano or microparticles (or in some instances larger) which consist in whole or in part of the peripheral opioid antagonists or other therapeutic agent(s) as described herein. The particles may contain the active ingredients in a core surrounded by a coating, including, but not limited to, an enteric coating. The active ingredients also may be dispersed throughout the particles. The active ingredients also may be adsorbed into the particles. The particles may be of any order release kinetics, including zero order release, first order release, second order release, delayed release, sustained release, immediate release, and any combination thereof, etc. The particle may include, in addition to the active ingredients, any of those materials routinely used in the art of pharmacy and medicine, including, but not limited to, erodible, nonerodible, biodegradable, or nonbiodegradable material or combinations thereof. The particles may be microcapsules which contain the antagonist in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as nonimmediate release formulations, with nonimmediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

In another embodiment, drug dosage forms are provided that comprise an enterically coated, osmotically activated device housing a formulation of the invention. In this embodiment, the drug-containing formulation is encapsulated in a semipermeable membrane or barrier containing a small orifice. As known in the art with respect to so-called “osmotic pump” drug delivery devices, the semipermeable membrane allows passage of water in either direction, but not drug. Therefore, when the device is exposed to aqueous fluids, water will flow into the device due to the osmotic pressure differential between the interior and exterior of the device. As water flows into the device, the drug-containing formulation in the interior will be “pumped” out through the orifice. The rate of drug release will be equivalent to the inflow rate of water times the drug concentration. Suitable materials for the semipermeable membrane include, but are not limited to, polyvinyl alcohol, polyvinyl chloride, semipermeable polyethylene glycols, semipermeable polyurethanes, semipermeable polyamides, semipermeable sulfonated polystyrenes and polystyrene derivatives; semipermeable poly(sodium styrenesulfonate), semipermeable poly(vinylbenzyltrimethylammonium chloride), and cellulosic polymers such as cellulose acetate, cellulose diacetate, cellulose triacetate, cellulose propionate, cellulose acetate propionate, cellulose acetate butyrate, cellulose trivalerate, cellulose trilmate, cellulose tripalmitate, cellulose trioctanoate, cellulose tripropionate, cellulose disuccinate, cellulose dipalmitate, cellulose acetate succinate, cellulose propionate succinate, cellulose acetate octanoate, cellulose valerate palmitate, cellulose acetate heptanate, cellulose acetaldehyde dimethyl acetate, cellulose acetate ethylcarbamate, cellulose acetate methylcarbamate, cellulose dimethylaminoacetate and ethylcellulose.

Enterically coated, osmotically activated devices can be manufactured using conventional materials, methods and equipment. For example, osmotically activated devices may be made by first encapsulating, in a pharmaceutically acceptable soft capsule, a liquid or semi-solid formulation as described previously. This interior capsule is then coated with a semipermeable membrane composition (comprising, for example, cellulose acetate and polyethylene glycol 4000 in a suitable solvent such as a methylene chloride-methanol admixture), for example using an air suspension machine, until a sufficiently thick laminate is formed, e.g., around 0.05 mm. The semipermeable laminated capsule is then dried using conventional techniques. Then, an orifice having a desired diameter (e.g., about 0.99 mm) is provided through the semipermeable laminated capsule wall, using, for example, mechanical drilling, laser drilling, mechanical rupturing, or erosion of an erodible element such as a gelatin plug. The osmotically activated device may then be enterically coated as previously described. For osmotically activated devices containing a solid carrier rather than a liquid or semi-solid carrier, the interior capsule is optional; that is, the semipermeable membrane may be formed directly around the carrier-drug composition. However, preferred carriers for use in the drug-containing formulation of the osmotically activated device are solutions, suspensions, liquids, immiscible liquids, emulsions, sols, colloids, and oils. Particularly preferred carriers include, but are not limited to, enterically coated capsules containing liquid or semisolid drug formulations.

In another embodiment, drug dosage forms are provided that comprise a sustained release coated device housing a formulation of the invention. In this embodiment, the drug-containing formulation is encapsulated in a sustained release membrane. The membrane may be semipermeable, as described above. Semipermeable membranes allow passage of water inside the coated device and then dissolve the drug. The dissolved drug solution then diffuses out through the semipermeable membrane. The rate of drug release therefore depends upon the thickness of the coated film and the release of drug can begin in any part of the GI tract. Suitable membrane materials include ethyl cellulose.

In another embodiment, drug dosage forms are provided that comprise a sustained release device housing a formulation of the invention. In this embodiment, the drug-containing formulation is uniformly mixed with a sustained release polymer. These sustained release polymers may be high molecular weight water-soluble polymers, which when contacted may be water, swell and create channels for water to diffuse inside and dissolve the drug. As the polymers swell and dissolve in water, more of drug is exposed to water for dissolution. Such a system is generally referred to as a sustained release matrix. Suitable materials for such a system include hydropropyl methylcellulose, hydroxypropyl cellulose, hydroxyethyl cellulose, and methyl cellulose.

In another embodiment, drug dosage forms are provided that comprise an enteric coated device housing a sustained release formulation of the invention. In this embodiment, the drug containing product described above coated with an enteric polymers. Such a device does not release any drug in the stomach. When the device reaches the intestine, the enteric polymer begins to dissolve and release the drug. The drug release may take place in a sustained release fashion.

Cellulose coatings include those of cellulose acetate phthalate and trimellitate; methacrylic acid copolymers, e.g. copolymers derived from methylacrylic acid and esters thereof, containing at least 40% methylacrylic acid; and especially hydroxypropyl methylcellulose phthalate. Methylacrylates include those of molecular weight above 100,000 daltons based on, e.g. methylacrylate and methyl or ethyl methylacrylate in a ratio of about 1:1. Typical products include EUDRAGIT L, e.g. L 100-55, marketed by Rohm GmbH, Darmstadt, Germany. Typical cellulose acetate phthalates have an acetyl content of 17-26% and a phthalate content of from 30-40% with a viscosity of ca. 45-90 cP. Typical cellulose acetate trimellitates have an acetyl content of 17-26%, a trimellityl content from 25-35% with a viscosity of ca. 15-20 cS. An example of a cellulose acetate trimellitate is the marketed product CAT (Eastman Kodak Company, USA). Hydroxypropyl methylcellulose phthalates typically have a molecular weight of from 20,000 to 130,000 daltons, a hydroxypropyl content of from 5 to 10%, a methoxy content of from 18 to 24% and a phthalyl content from 21 to 35%. An example of a cellulose acetate phthalate is the marketed product CAP (Eastman Kodak, Rochester N.Y., USA). Examples of hydroxypropyl methylcellulose phthalates are the marketed products having a hydroxypropyl content of from 6-10%, a methoxy content of from 20-24%, a phthalyl content of from 21-27%, a molecular weight of about 84,000 daltons, known under the trade mark HP50 and available from Shin-Etsu Chemical Co. Ltd., Tokyo, Japan, and having a hydroxypropyl content, a methoxyl content, and a phthalyl content of 5-9%, 18-22% and 27-35%, respectively, and a molecular weight of 78,000 daltons, known under the trademark HP55 and available from the same supplier.

The therapeutic agents may be provided in capsules, coated or not. The capsule material may be either hard or soft, and as will be appreciated by those skilled in the art, typically comprises a tasteless, easily administered and water soluble compound such as gelatin, starch or a cellulosic material. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals.

The therapeutic agents may be provided in suppositories. Suppositories are solid dosage forms of medicine intended for administration via the rectum. Suppositories are compounded so as to melt, soften, or dissolve in the body cavity (around 98.6.degree. F.) thereby releasing the medication contained therein. Suppository bases should be stable, nonirritating, chemically inert, and physiologically inert. Many commercially available suppositories contain oily or fatty base materials, such as cocoa butter, coconut oil, palm kernel oil, and palm oil, which often melt or deform at room temperature necessitating cool storage or other storage limitations. U.S. Pat. No. 4,837,214 to Tanaka, et al. describes a suppository base comprised of 80 to 99 percent by weight of a lauric-type fat having a hydroxyl value of 20 or smaller and containing glycerides of fatty acids having 8 to 18 carbon atoms combined with 1 to 20 percent by weight diglycerides of fatty acids (which erucic acid is an example of). The shelf life of these type of suppositories is limited due to degradation. Other suppository bases contain alcohols, surfactants, and the like which raise the melting temperature but also can lead to poor absorption of the medicine and side effects due to irritation of the local mucous membranes (see for example, U.S. Pat. No. 6,099,853 to Hartelendy et al., U.S. Pat. No. 4,999,342 to Ahmad, et al., and U.S. Pat. No. 4,765,978 to Abidi, et al.).

The base used in the pharmaceutical suppository composition of this invention include, in general, oils and fats comprising triglycerides as main components such as cacao butter, palm fat, palm kernel oil, coconut oil, fractionated coconut oil, lard and WITEPSOL®, waxes such as lanolin and reduced lanolin; hydrocarbons such as VASELINE®, squalene, squalane and liquid paraffin; long to medium chain fatty acids such as caprylic acid, lauric acid, stearic acid and oleic acid; higher alcohols such as lauryl alcohol, cetanol and stearyl alcohol; fatty acid esters such as butyl stearate and dilauryl malonate; medium to long chain carboxylic acid esters of glycerin such as triolein and tristearin; glycerin-substituted carboxylic acid esters such as glycerin acetoacetate; and polyethylene glycols and its derivatives such as macrogols and cetomacrogol. They may be used either singly or in combination of two or more. If desired, the composition of this invention may further include a surface active agent, a coloring agent, etc., which are ordinarily used in suppositories.

The pharmaceutical composition of this invention may be prepared by uniformly mixing predetermined amounts of the active ingredient, the absorption aid and optionally the base, etc. in a stirrer or a grinding mill, if required at an elevated temperature. The resulting composition may be formed into a suppository in unit dosage form by, for example, casting the mixture in a mold, or by forming it into a gelatin capsule using a capsule filling machine.

The compositions according to the present invention also can be administered as a nasal spray, nasal drop, suspension, gel, ointment, cream or powder. The administration of a composition can also include using a nasal tampon or a nasal sponge containing a composition of the present invention.

The nasal delivery systems that can be used with the present invention can take various forms including aqueous preparations, non-aqueous preparations and combinations thereof. Aqueous preparations include, for example, aqueous gels, aqueous suspensions, aqueous liposomal dispersions, aqueous emulsions, aqueous microemulsions and combinations thereof. Non-aqueous preparations include, for example, non-aqueous gels, non-aqueous suspensions, non-aqueous liposomal dispersions, non-aqueous emulsions, non-aqueous microemulsions and combinations thereof. The various forms of the nasal delivery systems can include a buffer to maintain pH, a pharmaceutically acceptable thickening agent and a humectant. The pH of the buffer can be selected to optimize the absorption of the therapeutic agent(s) across the nasal mucosa.

With respect to the non-aqueous nasal formulations, suitable forms of buffering agents can be selected such that when the formulation is delivered into the nasal cavity of a mammal, selected pH ranges are achieved therein upon contact with, e.g., a nasal mucosa. In the present invention, the pH of the compositions should be maintained from about 2.0 to about 6.0. It is desirable that the pH of the compositions is one which does not cause significant irritation to the nasal mucosa of a recipient upon administration.

The viscosity of the compositions of the present invention can be maintained at a desired level using a pharmaceutically acceptable thickening agent. Thickening agents that can be used in accordance with the present invention include methyl cellulose, xanthan gum, carboxymethyl cellulose, hydroxypropyl cellulose, carbomer, polyvinyl alcohol, alginates, acacia, chitosans and combinations thereof. The concentration of the thickening agent will depend upon the agent selected and the viscosity desired. Such agents can also be used in a powder formulation discussed above.

The “RDI for vitamins and minerals” refers to the list published in the 1993 Federal Register, Vol. 58. RDIs are a set of dietary references based on the Recommended Dietary Allowances (RDA) for essential vitamins and minerals. The name “RDI” replaces the term, “U.S. RDA” (Recommended Daily Allowances). Recommended Dietary Allowances (RDA) are the set of estimated nutrient allowances established by the National Academy of Sciences used as the basis for setting the U.S. RDAs. It is updated periodically to reflect current scientific knowledge.

Vitamins and minerals can be included in the product of the invention such that a significant proportion of the Reference Daily Intake (RDI) is supplied in a serving. In one embodiment of the invention, a single serving of the product can provide about 50% of the RDI for calcium, about 35% of the RDI for vitamin A, vitamin D, vitamin K, riboflavin, vitamin B.sub.6, vitamin B.sub.12, pantothenic acid, iodine, zinc, copper, chromium, vitamin C, iron, vitamin E, thiamin, niacin, folate, biotin, phosphorus, selenium, manganese and molybdenum, and about 15% of the RDI for magnesium.

The product can also contain other ingredients such as one or a combination of other vitamins, minerals, antioxidants, fiber, micronutrients and herb supplements (e.g., ginkgo biloba, ginseng, beta-carotene, inositol) and other nutritional supplements. Selection of one or several of these ingredients is a matter of formulation design, consumer and end-user preference. The amounts of these ingredients added to the products of this invention are readily known to the skilled artisan.

### Timing and Frequency of Administration

An effective amount, as herein defined, of the composition to be administered pursuant to embodiments of the invention is the most preferred method of expression of dosage. As known by those of ordinary skill in the art, preferred dosages of the present components of the present invention/compositions will vary and may dependent on many factors, including but not limited to, the type and severity of the condition or trauma being treated or prevented, the patient's general health, size, age, gender, weight, and the nature of treatment, e.g., short-term or chronic treatment.

Generally, the treatment may be given in a single dose or multiple administrations. Outside of the surgical context, however, it is often impossible to know when an instance of TBI or other CNS trauma will be encountered. As such, most preferably, a plurality of doses is administered to the individual over a treatment period, e.g. a period beginning prior to anticipated CNS trauma, during, and/or after the infliction of such trauma, for enhanced prophylactic and/or palliative effects. In some embodiments, the composition is administered once, twice, three or more times daily over a period of time. In other embodiments, the composition is administered continuously by methods known in the art. As one of ordinary skill will recognize such variations are largely dependent upon factors such the severity of the condition being treated, or prevented, the dosage required for therapeutic efficacy, and/or the age, gender, weight, and health state of the individual.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Formulations: Two versions of the formula were tested in a rat model of TBI and compared to those of a placebo treated control group. The primary outcome variables were reduced behavioral deficits as shown in the accompanying Morris Water Maze figure.

Three groups of rats received (by oral gavage) either a test formula or a calorie equivalent dosage of sucrose water as a control. In Test Group I, the fifteen (15) Sprague Dawley rats were given one cc of the test formula every day for seven days prior to receipt of TBI and the same dosage again one day (24 hours) post TBI every day until the completion of their testing. In Test Group II, another group of fifteen (15) Sprague Dawley rats were given one cc of the test formula every day starting 24 hours after receiving the TBI. Test Group III was made up of another group of 16 Sprague Dawley rats. The dosage has been calculated as the rat equivalent of 8 fl oz (or 237 ml) for a 70 kg person (237 ml/70 kg=3.4 ml/kg), thus a 300 gram rat will receive about 1.02 ml (3.4 ml/kg X 0.3 kg).

TBI MODEL: The rat lateral fluid percussion model is the most extensively characterized TBI model and is used extensively to evaluate potential therapeutics. The device consists of a Plexiglas cylindrical reservoir filled with dH₂O. A piston is fitted into one end of the cylinder, with the other end equipped with a pressure transducer and male Luer-Loc syringe fitting. Brain injury was produced in the rats by a metal pendulum striking the piston which injects a small volume of saline epidurally into the closed cranial cavity, producing a brief displacement and deformation of the underlying brain. The resulting pressure pulse is measured an extracranial transducer and recorded on a storage oscilloscope.

### Example 2

Histology: Rats will be anesthetized with pentobarbital and transcardially perfused with sodium phosphate buffer followed by 4% paraformaldehyde. The brains removed, blocked and post-fixed in the same fixative solution overnight at 4° C. Serial coronal vibratome sections containing dorsal hippocampus at 30 μM thick are mounted on slides and stained with cresyl-violet. Slides will be coded and blinded to for the counting technician.

### Example 3

Stereological Cell Counts: Unbiased techniques will estimate the number of surviving hippocampal CA3 pyramidal neurons. Cell counting will be performed using the optical fractionator stereological method by calculating the total number of cells in the entire reference space by summing the objects sampled in the individual dissectors and multiplying by the reciprocals of the sampling fractions for section, area, and thickness of the reference space. This procedure insures that each cell of interest within the reference space has an equal probability of being sampled and that each cell can be sampled only once.

### Example 4

Behavioral Evaluation: Spatial learning/memory performance of each individual animal in the three groups was assessed with a Morris water maze task on days 10-14 after TBI. Three data sets were acquired: 1) a visible platform task to detect visual impairment, 2) hidden platform acquisition to assess spatial learning and memory, and 3) a probe trial without an escape platform to assess memory retention. The test apparatus was a circular tank (183 cm dia.) filled with 26±2° C. water, and with consistent visual cues. Rats received 4 trials/day over 5 consecutive days recorded using a video tracking system. Time to find and mount the submerged platform and distance swum were recorded. The testing technician was blind to the identity of the animal's test group.

### Example 5

Statistics: A power analysis determined that a sample size of n=15 per group is necessary to achieve 80% statistical power with a set to p<0.05. Data was analyzed using ANOVA (histology) and repeated measures ANOVA (behavior) and Bonferroni post-hoc test. Alpha level for Type I error is set at 0.05 for rejecting the null hypotheses. Below is a table showing the latency of percent change with respect to the individual rats baseline. As indicated by the first point in the graph of the accompanying FIGURE, the baseline was set as the performance at day 11 after traumatic brain injury. The mean values represent the mean of all of the animals in a particular group.

Report

| Group | | Day 12 | Day 13 | Day 14 | Day 15 |
|---|---|---|---|---|---|
| Test Formula pre-post | Mean | −28.2522 | −49.8562 | −71.3237 | −78.6077 |
| | N | 15 | 15 | 15 | 15 |
| | Std. Deviation | 28.21530 | 29.30696 | 18.17286 | 13.91752 |
| | Std. Error of Mean | 7.28516 | 7.56703 | 4.69221 | 3.59349 |
| Test Formula post | Mean | −23.7577 | −45.0496 | −63.9415 | −70.9815 |
| | N | 15 | 15 | 15 | 15 |
| | Std. Deviation | 63.09672 | 38.20691 | 30.68011 | 14.80791 |
| | Std. Error of Mean | 16.29150 | 9.86498 | 7.92157 | 3.82339 |
| Calorie control | Mean | −4.3532 | −36.8569 | −53.5802 | −65.2426 |
| | N | 16 | 16 | 16 | 16 |
| | Std. Deviation | 26.99132 | 30.39959 | 22.73183 | 17.33726 |
| | Std. Error of Mean | 6.74783 | 7.59990 | 5.68296 | 4.33431 |
| Total | Mean | −18.4739 | −43.7673 | −62.7448 | −71.4722 |
| | N | 46 | 46 | 46 | 46 |
| | Std. Deviation | 42.90907 | 32.54679 | 24.95401 | 16.10975 |
| | Std. Error of Mean | 6.32660 | 4.79876 | 3.67927 | 2.37525 |

### Example 6

To further illustrate the present invention, the following are two exemplary formulations based on the serving size of about 8 fluid ounces of beverage:
Formula I=pomegranate juice 4 oz., blueberry juice 4 oz., magnesium citrate 5 ml, curcumin 250 mg.
Formula II=pomegranate juice 4 oz., blueberry juice 4 oz., magnesium citrate 5 ml, curcumin 250 mg, and Omega 3 fatty acids 300 mg.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A composition comprising pomegranate extract, blueberry extract, and curcumin.

2. The composition of claim 1, further comprising magnesium citrate.

3. The composition of claim 1 or 2, further comprising Omega 3 fatty acids.

4. The composition of claim 1, further comprising a food, food-forming, beverage, or beverage-forming component to formulate an edible composition.

5. A beverage useful for providing neuroprotection in an individual, comprising water, pomegranate extract, blueberry extract, and curcumin.

6. The beverage of claim 5, further comprising magnesium citrate.

7. The beverage of claim 5 or 6, further comprising Omega 3 fatty acids.

8. The beverage of claim 5, said beverage being in carbonated form.

9. The beverage of claim 5, said beverage being storage-stable.

10. The beverage of claim 5, said beverage having a pH of from about 2 to about 8.

11. The beverage of claim 5, said beverage further comprising a neuroprotective agent.

12. The beverage of claim 5, further comprising a sweetening agent.

13. The beverage of claim 12, wherein the sweetening agent comprises a natural sweetener.

14. The beverage of claim 12, wherein the sweetening agent comprises a non-nutritive sweetener.

15. The beverage of claim 14, wherein the non-nutritive sweetener is selected from the group consisting of saccharin, cyclamate, aspartame, and acesulfame potassium.

16. The beverage of claim 5, further comprising a preservative.

17. The beverage of claim 14, wherein said preservative is selected from the group consisting of potassium sorbate, sodium benzoate, quaternary amine, and methylparaben.

18. The beverage of claim 5, wherein said one or more extracts is a deflavored, decolored extract.

19. The beverage of claim 5, further comprising an edible acid.

20. The beverage of claim 19, wherein said edible acid is selected from the group consisting of phosphoric, citric, malic, tartaric, lactic, formic, ascorbic, isoascorbic, hydrochloric, sulfuric, fumaric, and adipic.

21. The beverage of claim 5, further comprising an emulsifier.

22. The beverage of claim 21, wherein the emulsifier is a food-grade emulsifier.

23. The beverage of claim 5, further comprising a surfactant.

24. The beverage of claim 5, further comprising caffeine.

25. The beverage of claim 5, further comprising nutrient fortification.

26. The beverage of claim 5, further comprising a coloring agent.

27. The beverage of claim 5, further comprising an herb.

28. The beverage of claim 5, further comprising a spice.

29. The beverage of claim 5, further comprising a foam reducer.

30. A method of conferring neuroprotection to a population of cells in an individual, comprising administering to the individual a composition comprising pomegranate extract, blueberry extract, and curcumin.

31. The method of claim 30, wherein said composition further comprises magnesium citrate.

32. The method of claim 30, further comprising administering Omega 3 fatty acids.

33. The method of claim 32, wherein the composition and Omega 3 fatty acids are administered from separate compositions.

34. The method of claim 30, wherein said composition further comprises water.

35. The method of claim 30, wherein said composition is administered orally.

36. The method of claim 30, wherein the composition is a liquid composition.

37. The method of claim 30, wherein the individual is at risk for ischemic injury to the CNS.

38. The method of claim 30, wherein the individual is at risk for CNS trauma.

39. The method of claim 38, wherein the individual is at risk for trauma to the spine.

40. The method of claim 38, wherein the CNS trauma is TBI.

41. The method of claim 30, further comprising the individual undergoing a surgical procedure.

42. The method of claim 41, comprising the individual undergoing a surgical procedure after the administration step.

43. The method of claim 30, further comprising the individual undergoing a neurological test.

44. The method of claim 43, comprising the individual undergoing a neurological test after the administration step.

45. A method of reducing serum cholesterol comprising administering to the individual in need thereof a composition comprising pomegranate extract, blueberry extract, and curcumin.

46. The method of claim 45, wherein said composition further comprises water.

47. The method of claim 45, further comprising the individual undergoing a diagnostic for serum cholesterol.

48. The method of claim 47, comprising the individual undergoing the diagnostic after the administration step.
